(19) United States
(12) Patent Application Publication
(10) Pub. No.: US 2010/0166913 A1
(43) Pub. Date: Jul. 1, 2010

(54) CELLULOSIC ETHANOL DISTILLERS RESIDUES

(76) Inventor: DAVID A. STEWART, Boca Raton, FL (US)

Correspondence Address: FLEIT GIBBONS GUTMAN BONGINI & BIANCO P.L., ONE BOCA COMMERCE CENTER, 551 NORTHWEST 77TH STREET, SUITE 111, BOCA RATON, FL, 33487, US

(21) Appl. No.: 12/643,510

(22) Filed: Dec. 21, 2009

Related U.S. Application Data
(60) Provisional application No. 61/139,268, filed on Dec. 19, 2008.
(60) Provisional application No. 61/139,217, filed on Dec. 19, 2008.
(60) Provisional application No. 61/139,360, filed on Dec. 19, 2008.

(51) Int. Cl. A23K 1/00; C12P 1/00; C10L 1/18

(52) U.S. Cl. 426/54; 426/635; 435/41; 44/313

(57) ABSTRACT

Biomass from ethanol whole stillage and processes for making same are disclosed. The process overcomes the challenge of dewatering the stillage through the use of a press aid or a mixture of the stillage and unfermented biomass. The invention further provides a system and method for producing distillers peels pellets from fermented citrus waste. The invention allows the composition characteristics of the animal feed or bio fuel to be varied and optimized by controlling the mix proportions of the feedstock.

### CROSS-REFERENCE TO RELATED APPLICATIONS

This application is based upon and claims priority from prior U.S. Provisional Patent Application No. 61/139,268, filed on Dec. 19, 2008, the entire disclosure of which is herein incorporated by reference. This Application is related to co-pending U.S. Provisional Patent Application No. 61/139,217 and U.S. Provisional Patent Application No. 61/139,360 each filed on Dec. 19, 2008, the entire disclosure of which each is herein incorporated by reference.

### FIELD OF THE INVENTION

This invention relates to a novel animal feed and biofuel made from citrus waste and methods of producing same.

### BACKGROUND OF THE INVENTION

Citrus waste consists primarily of peel, membranes, and seeds, which result from processing citrus fruit for juice. Approximately 5 million tons of citrus waste are produced each year in Florida alone. Most of this peel waste is dried, pelletized, and sold as a beef or milk cattle feed filler known as citrus pulp pellets (“CPP”).

In a typical cattle feeding program, citrus pulp pellets serve as a bulk carbohydrate energy feed with a high degree of water absorption, an above average palatability for cattle, and a high total digestible nutrient content. As a general rule, 40-45% of the ground snapped corn in a dairy ration can be replaced by dried citrus pulp pellets.

Unfortunately, citrus pulp pellet production can have a significant adverse environmental impact. For example, citrus pulp pellet feed mills often emit a significant amount of volatile organic compounds (“VOCs”), including an orange scented oil present in the citrus rind called limonene. Although in some cases thermal oxidizers or other equipment can be installed to reduce the VOCs emissions to acceptable levels, such equipment is typically expensive to install and operate. For this reason, processes that can produce citrus pulp pellets with minimum VOC emissions are desirable.

The global energy crisis, coupled with the effect fossil fuels are having on the environment, have led to continuing research in the area of alternative fuels. An attractive alternative is biomass fuels, such as ethanol. Ethanol produced from biomass is referred to as “cellulosic ethanol” and is usually defined as fuel ethanol produced from non-food crops such as agricultural residues (e.g., citrus waste, wheat straw, corn stover, bagasse, beet pulp, apple pommaceo, and corn husks), woody materials (e.g., hurricane debris, sawdust, soft wood, hard wood, and forestry waste), energy crops (e.g., switch grass, canes, and poplar trees) and waste materials like Municipal Solid Waste, MSW.

A significant amount of research is being directed to producing ethanol from citrus waste. Citrus waste contains, among other things, several mono and disaccharides, mainly glucose, sucrose and fructose. Citrus waste also contains the polysaccharides cellulose, hemicellulose and pectin (Ting and Deszyck, 1961). Cellulose, hemicellulose and pectin can be hydrolyzed using a cocktail of pectinase, cellulase, and beta-glucosidase enzymes to produce glucose, fructose, arabinose, xylose, galactose, rhamnose, and galacturonic acid (GA) (Nishio and Nagai, 1979; Marshall et al., 1985; Ben-Shalom, 1986; Echeverria et al., 1988; Grohmann and Baldwin, 1992; Grohmann et al., 1994, 1995). Fructose, glucose, sucrose and galactose can be fermented by Saccharomyces cerevisiae yeast (typically used in the brewing industry) to produce ethanol (Grohmann et al., 1994).

Accordingly, a standard process for generating ethanol from citrus waste can be envisioned as two principal chemical transformations. The first is the hydrolysis of the complex polysaccharides in the citrus peel to fermentable sugars, a process referred to as saccharification. The second involves the conversion of the fermentable sugars to ethanol using yeast or some other fermenting microorganism, a process referred to as fermentation. The process also typically involves removing limonene prior to fermentation to avoid its inhibitory effects.

Following these transformations, the ethanol may be recovered by stripping or other means and a whole stillage remains as a by-product. There is some disclosure in the literature by Stewart et al., US Patent Application No. 2006/0177916 of using this whole stillage to make cattle feed. However, this disclosure does not include any protocol, results or data indicating whether such a process is feasible using traditional techniques, whether the resultant cattle feed has a nutritional value comparable to CPP, or whether the process is economically viable.

It has been subsequently discovered that the preparation of citrus pellets from 100% distillers peels (“DPP”) using traditional means produces pellets that break into small particles (or dust) and fail to hold together. An additional problem is that the whole stillage must be dewatered in order to be efficiently transported and have a reasonable “shelf life.”

In view of the foregoing, a problem that must be overcome is that existing distilled peel pellets break down when handled over time and improper preparation can lead to spoilage or spontaneous combustion. The distilled peel pellets produced from the whole stillage must also serve as an adequate replacement for citrus pulp pellets and be compatible with the existing citrus pulp pellet feedmill equipment. The present invention is directed to these, as well as other, important ends.

### SUMMARY OF THE INVENTION

It is therefore an object of the present invention to provide novel animal feeds and biofuels.

It is a further object of the invention to provide animal feed and biofuel with improved qualities and compositions.

It is a further object of the invention to provide new methods of producing animal feed and biofuel.

It is a further object of the invention to provide methods for reducing the amount of VOCs emitted in the process for making animal feed and biofuel.

It is a further object of the invention to provide compositions and methods relating to the process of producing ethanol from citrus waste.

These and other aspects of the present invention will be further disclosed in the following description of the drawings and preferred embodiments.

### BRIEF DESCRIPTION OF THE DRAWINGS

FIG. 1 is a simplified flow schematic of the process of one embodiment of the invention.

### DETAILED DESCRIPTION OF THE PREFERRED EMBODIMENTS

Thus, in a first embodiment, the present invention provides a biomass comprising:

Carbohydrates between about 55% to about 65%;

Fiber between about 8% to about 13%;

Protein between about 4% to about 10%;

Fat between about 1% to about 7%;

Ash between about 5% to about 12%; and

Water between about 8% to about 12%.

In certain embodiments, the biomass is produced from an ethanol whole stillage with the use of a press aid. In certain embodiments, the press aid is a fibrous material such as wood, straw, bagasse, pulp, cellulose, etc.

In certain embodiments, the biomass is produced through the addition of unfermented citrus peel to an ethanol whole stillage. In certain embodiments, the allocation of fermentation versus non-fermentation material is 90%-10%, 80%-20%, 70%-30%, 60%-40%, 50%-50%, 40%-60%, 30%-70%, 20%-80% and 10%-90%, respectively. In certain embodiments, the percent allocation is dynamically controllable. In certain embodiments, the biomass production results in reduced emissions of VOCs compared to the emissions resulting from the preparation of CPP.

In certain embodiments, the biomass serves as an animal feed. In certain embodiments, the biomass serves as a biofuel.

In certain embodiments, the present invention provides a method for producing a biomass, the method comprising (a) providing an ethanol whole stillage; (b) adding a press aid to the ethanol whole stillage to form a wet biomass; and (c) pressing the wet biomass to provide a pressed biomass.

In certain embodiments, the press aid is added to the ethanol whole stillage in a mix tank or pug mill. In certain embodiments, the ethanol whole stillage is centrifuged before the press aid is added. In certain embodiments, the centrifuge results in a supernatant that is subjected to an evaporator. In certain embodiments, the wet biomass is pressed with a mechanical press. In certain embodiments, the pressing provides a press liquor that is subjected to an evaporator. In certain embodiments, the pressed biomass is dried to produce a dry biomass. In certain embodiments, the dried biomass is pelletized. In certain embodiments, the dried biomass has a low moisture content. In certain embodiments, the dried biomass serves as an animal feed or biofuel.

In certain embodiments, the present invention provides a method for producing a biomass, the method comprising (a) providing an ethanol whole stillage; (b) adding unfermented citrus waste to the ethanol whole stillage to form a mixture of fermented and unfermented biomass; and (c) pressing the mixture to provide a mixed biomass.

In certain embodiments, the unfermented citrus waste is added to the ethanol whole stillage in a mix tank or pug mill. In certain embodiments, the ethanol whole stillage is centrifuged before the unfermented citrus waste is added. In certain embodiments, the mixed biomass is pressed in a mechanical press. In certain embodiments, the mixed biomass is dried to produce a dry mixed biomass. In certain embodiments, the preparation of the dry mixed biomass results in reduced emissions of VOCs compared to the emissions resulting from the preparation of CPP. In certain embodiments, the dry mixed biomass is pelletized. In certain embodiments, the mixed biomass is used to make pellets having low moisture content. In certain embodiments, the biomass serves as an animal feed or biofuel.

In certain embodiments, the ethanol whole stillage is derived from a fermentation process. In certain embodiments, the ethanol whole stillage is derived from a sequential simultaneous saccharification and fermentation process. In certain embodiments, the ethanol whole stillage is derived from a simultaneous saccharification and fermentation process. In certain embodiments, the ethanol has been removed from the ethanol whole stillage through stripping, distillation, evaporation or the like.

In certain embodiments, the ethanol whole stillage is derived from citrus peel waste. In certain embodiments, the citrus peel waste undergoes a pre-treatment stage. In certain embodiments, the pre-treatment stage is selected from manipulation by mechanical means, the application of heat, steam explosion, and the addition of chemicals. In certain embodiments, the citrus peel waste undergoes limonene removal. In certain embodiments, the limonene removal is performed prior to fermentation.

In certain embodiments, a dewatering agent is employed. In certain embodiments, the dewatering agent is lime. In certain embodiments, the dewatering agent is added to a mix tank or pug mill. In certain embodiments, the dewatering agent is added prior to a pre-treatment step.

As noted above, the present invention overcomes several problems associated with the prior art, including issues associated with the existing production of CPP and DPP. In one respect, the present invention minimizes or eliminates the need to dewater the whole stillage through an efficient and economic process that allows the production of an animal feed or biofuel in pellet or flake form. These pellets or flakes have sufficient adhesion characteristics to demonstrate a suitable shelf-life and a low enough moisture content to prevent spoilage or spontaneous combustion. Unexpectedly, these new pellets or flakes maintain their nutritional content compared to traditional CPP.

The invention capitalizes, in part, on the discovery that the addition of a press agent to an “ethanol whole stillage,” “whole stillage” or simply “stillage” (i.e., the liquid and/or material following ethanol removal) produces citrus pulp pellets that maintain their structural integrity over time. The resulting animal feeds or biofuels therefore have improved qualities and composition compared to DPP.

The invention further capitalizes on the discovery that the addition of unfermented citrus peel to whole stillage produces animal feeds or biofuels having improved qualities over DPP. Where unfermented citrus peel is added to the whole stillage, a press agent may be used, but is not necessary. Significantly, the composition of the animal feed or biofuel may be varied and optimized by controlling the mix proportions of the fermented and unfermented biomass to produce a desired composition. The amount of unfermented peel largely determines how well the pellets hold together, and hence, may have a profound effect on their shelf life and other properties. As a general rule, the more unfermented biomass present, the better the quality of the resulting pellets. Thus, by controlling the mix proportions of the fermented and unfermented biomass, the invention controls the pellets' physical properties and allows them to be optimized in addition to the compositional properties.

It will also be appreciated that the fermented component of the biomass feedstock will typically have significantly reduced sources of VOCs. This can be the result of the pretreatment process, the action of the fermenting organism that reduces the sources of VOCs, and/or the stripping action of the CO₂ during fermentation. Accordingly, the level of VOC emissions can be reduced to a desirable level by mixing the fermented and unfermented biomass entering the dryer. In this manner, the improved methods of the present invention are able to produce distilled peel pellets with nutritional value and shelf life comparable to citrus peel pellets, but with decreased VOCs emissions.

A further advantage is the partial solid/liquid separation of the whole stillage using a centrifuge, or equivalent technique, preferably prior to feeding the mechanical press to allow flexibility in processing. A further advantage is the ability to control the efficiency of the pelletizer die and the properties of the pellets by controlling many of the operating conditions such as pressure, time, temperature and moisture. A further advantage is having the majority of the dewatering be done by mechanical systems versus direct or indirect heating systems.

With respect to the process, a biomass such as citrus waste is generally fed into a reaction system of suitable equipment. The biomass may be reduced in particle size if necessary using a hammer mill or other similar means. The biomass may be split into two streams; stream one going to the pre-treatment stage for ethanol production; stream two going directly to a mix tank, pug mill, etc. The percentage of the biomass in each stream can be modified from 0 to 100%, including every increment between, to optimize the mix of final products.

Stream one is preferably pretreated by mechanical, heat, chemical, or some combination thereof prior to being hydrolyzed (typically by acid or enzymes) and then fermented separately or simultaneously. Ethanol may be recovered from the biomass beer using a membrane filter or beer stripper, etc. and the residue is referred to herein as the “ethanol whole stillage” or “whole stillage.” In some situations, depending on the characteristics of the whole stillage, it may then be subjected to a partial solid/liquid separation in a centrifuge. Where a centrifuge or equivalent is used, the supernatant output of the centrifuge may go to an evaporator and the solid stream from the whole stillage would move to a mix tank, pug mill, mix conveyor, or equivalent, where it may be combined with the unprocessed stream (stream two).

Where a particular pellet composition is desired, the unprocessed material (stream two) and the whole stillage residue or solid stream from the whole stillage may be combined and mixed together accordingly. Such mixing provides an animal feel or biofuel with enhanced composition and qualities. A dewatering agent, such as lime with citrus waste, may be added in the mix tank or earlier in the process. The reaction may be time controlled by time in the mix tank or pug mill.

In some embodiments, a “press aid,” which is typically a fibrous material from wood, straw, bagasse, pulp, etc. may be added as necessary. The mix may be passed into a mechanical press where press liquor may be separated from press cake. Press aids may be used with 100% of the whole stillage (stream one) from the ethanol process to produce an animal feed or biofuel with improved qualities compared those prepared in the absence of the press aid. Otherwise, the press aid may or may not be used with the mixed streams.

The press liquor may go to an evaporator and the press cake to the dryer. The dryer is typically fired with natural gas, oil, or biomass, and the waste heat is sent to an evaporator. The level of VOCs emission from the dryer exhaust can be adjusted by controlling the mix of fermented (from stream one) and unfermented (from stream 2) biomass entering the dryer. In some embodiments, after the material has been dried to suitable moisture content, it may be pelletized for future use as an animal feed or biomass fuel. Similarly, by controlling the mix proportions of the fermented and unfermented biomass put into the mixture the pellet physical properties can be controlled and optimized in addition to its compositional properties.

In some embodiments, the evaporator may be used to remove water from the supernatant, press liquor, and other liquid streams. The evaporated material may result in a molasses and this molasses material may be used in several parts of the process where it may enhance the function of the equipment and the energy content of the output pellets. Additionally, the evaporator may be used to capture volatile components in the ethanol whole stillage such as oils.

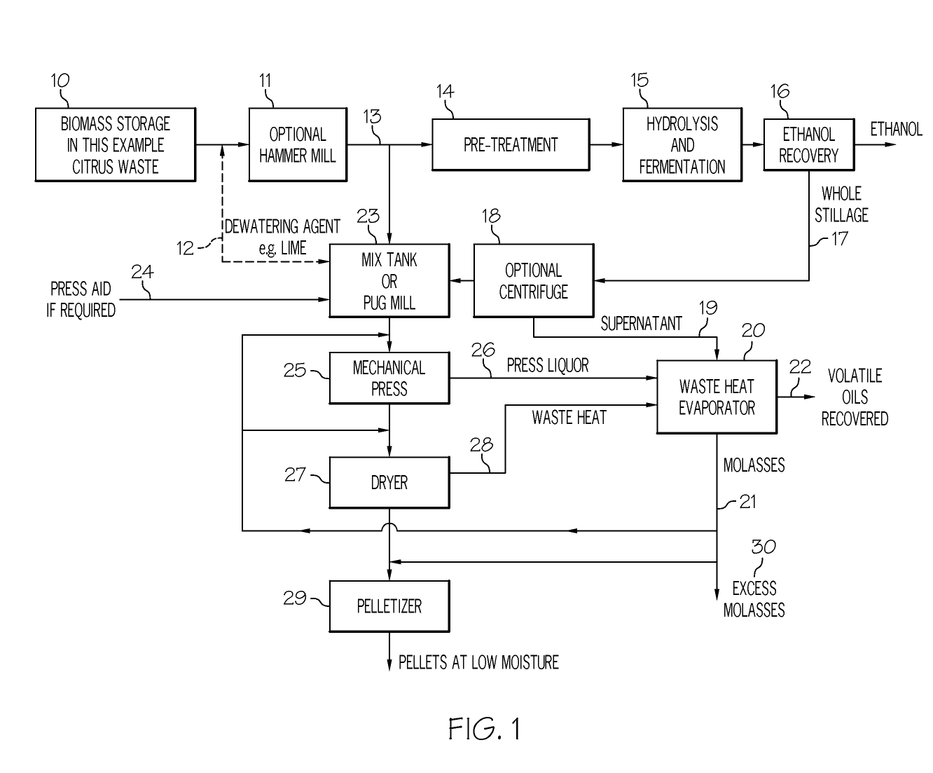

With reference to FIG. 1, biomass, preferably citrus waste, is conveyed or pumped from the local source 10. A dewatering agent 12 such as lime may be added to enhance mechanical dewatering at later stages and/or to give increased reaction time for the dewatering agent. In some embodiments, an optional sizing operation 11, may be performed by using a hammer mill, or its equivalent. The biomass may then be carried forward 13 in its entirety (as stream one), or alternatively, split into two streams (streams one and two).

In those embodiments where the streams are split, the allocation of material to each stream can be any percentage and may depend on the particular application. In certain preferred embodiments, the allocation may be 90%-10%, 80%-20%, 70%-30%, 60%-40%, 50%-50%, with respect to either stream. Such values are provided for general guidance and it is understood that any numerical endpoint or combination of endpoints is possible. In certain preferred embodiments, the percentage allocation is dynamically controllable. It will be appreciated that economic market conditions, as well as considerations relating to the desired qualities and consistencies of the end product, the desired use of a press agent, and other factors play a role in determining the optimal allocation. It is also understood that the sizing operation 11 can also be implemented after the streams are split. It is also understood that the biomass put into each stream may come from the same source or from different sources.

Several processes have been developed to convert biomass, such as citrus waste, into ethanol. For example, Stewart, et al., U.S. Publication No. 2006/0177916, incorporated herein by reference in its entirety, discloses a well-known method of producing an ethanol residue (whole stillage) that may be employed to arrive at this stage of the process. One of skill in the art will recognize, however, that any process which produces ethanol from any starting biomass and leaves whole stillage can be employed in the present invention.

By way of example, 60% of the citrus waste is sent to the process which will produce cellulosic ethanol. This portion of the citrus waste may enter a pre-treatment stage 14. The pre-treatment stage 14 may include mechanical means, the application of heat, steam explosion, addition of chemicals, or some combination thereof to prepare the material for subsequent reaction.

The pre-treatment stage may further include conditions to separate and/or remove citrus oil, such as D-limonene, from the mixture prior to hydrolysis and fermentation. This may be done either with steam stripping apparatus or using a steam autoclave to remove the citrus oil by volatilization. Citrus oil content of the material may be determined before and after pretreatment using the Scott oil method. The citrus oil level is preferably reduced during pretreatment to a level below 0.08% by mass to prevent fermentation inhibition.

Where the citrus waste undergoes steam stripping by discharge under pressure to a flash tank it is not necessary to comminute the material as the steam explosion results in a suitable particle size reduction. However, where an autoclave or equivalent is used to volatilize the citrus oils then the citrus waste has been comminuted by a food processor, or equivalent, to a particle size of a half inch or less.

The biomass is next preferably hydrolyzed 15, typically by acid or enzymes, a process which increases the amount of fermentable material. Fermentation 15 may be performed simultaneously with the hydrolysis, after hydrolysis, or without hydrolysis. The fermenting organisms will convert the fermentable material, typically five and six carbon sugars, into ethanol and by-products.

The saccharification (hydrolysis) and fermentation (“SSF”) may be carried out in rotating bottles with a CO₂ vent or in stirred fermenters. The conditions are typical for fermentations and typically include: sterilation equipment prior to SSF; maintaining pH levels between about 4.5 and about 5.5 by the addition of calcium carbonate as required; adding enzyme cocktails; maintaining the temperature between about 95 and about 100° F.; using an initial yeast concentration of about 0.7 g cells/100 g wet peel; continuously stirring or rotating of the material at several rpm; maintaining anaerobic conditions with CO₂ vent; and maintaining approximately atmospheric pressure. Where microbial contamination cannot be controlled because of difficulties cleaning equipment then chloramphenicol and cyclohexamide may be added at the level of 30 μg/mL.

The SSF process is typically allowed to proceed for about 24 hrs, after which the materials are deemed to be fermented “beer”. The beer may be passed directly to the ethanol removal stage in order that the enzymatic hydrolysis and associated decreasing viscosity are arrested by the denaturing of the enzymes as a result of a temperature above 200° F.

The ethanol, and by-products of interest, are then recovered 16 typically using a membrane filter, beer stripper, or equivalent, and the resulting residue is the whole stillage 17. Generally, after the ethanol is recovered, the fermentation residue, distillers peels, or whole stillage, may be passed through a machine press and then dried.

The whole stillage 17 will have various physical and chemical properties depending on the biomass and treatments used. In certain embodiments it will be economically beneficial to do a partial solid/liquid separation with a centrifuge, filter, or equivalent 18. Where a solid/liquid separation is utilized, the more liquid stream 19, supernatant in the case of a centrifuge, may be sent to a concentration device, such as an evaporator 20, where it may be concentrated into a high solids stream, such as molasses 21. Depending on the whole stillage, it may be economically worthwhile recovering some of the volatile components 22 from the whole stillage in the evaporator. In embodiments using citrus waste as the biomass, for example, there may be citrus oil, primarily d-limonene, available in useful quantities.

In certain embodiments, the whole stillage 17, or the solids from centrifuge 18, may be mixed with the biomass from the unprocessed stream (e.g., 40% of the original biomass in this example) from the split 13. The mixing apparatus 23 can be in a reaction tank, mixer tank, pug mill, mixer conveyor, or equivalent. In some embodiments, dewatering agents 12, such as lime, can be added and given the appropriate reaction time inside the mixing apparatus 23.

In certain embodiments, press aid 24 may be added to enhance the pressing operation. The press aid 24 may typically be a fibrous material from wood, straw, bagasse, or equivalent that helps the mechanical press 25 screens separate the solids and helps the screw move the material through the press and avoid backflow through the flights and mechanical interrupters. The press aid is necessary in embodiments that send 100% of the biomass through the fermentation process (e.g., that do include unfermented biomass in the mixture) to produce material that sufficiently holds together. As the percentage of unfermented biomass in the mix is increased, less press aid is needed.

The mixture may then be passed into a mechanical press 25, where press liquor 26 is separated from the remaining press cake. The press liquor 26 may be sent to an evaporator and concentrated in a similar manner to the supernatant 19. By controlling the portions of treated and untreated biomass, dewatering agents, press aid, and mechanical press 25 operating conditions, the moisture content of the press cake can be set to maximize the energy balance of the dryer 27 and the waste heat evaporator 20. In certain preferred embodiments the moisture content of material going into the dryer 27 would be in the range of about 50 to about 75%. This will allow the waste heat 28 being generated from the dryer 27 to power the waste heat evaporator 20 with enough heat to evaporate the liquids fed to the evaporator to a suitable solids concentration, typically 30 to 60%, without the addition of a second source of heat.

The moisture content of the untreated citrus waste and the wet distillers peels has been determined by drying at about 160° F. for about 20 hrs followed by vacuum drying at about 160° F. for about 1 hr, or as needed depending on the desired moisture level.

The dried press cake may be used as a food or energy source. The dried press cake may be used as an animal feed, such as cattle feed. Alternatively, the material may be burned as a bio fuel, for example, in a boiler. In certain preferred embodiments, press cake may then be passed into a pelletizer 29, and made in to pellets. The moisture at which these pellets are made can be controlled as desired, but is generally a low percentage.

The evaporated material 21, such as molasses, may be optionally used in several parts of the process where it may enhance the function of the equipment. In FIG. 1, for example, molasses 21 may be added prior to the mechanical press 25, prior to the dryer 27, or prior to the dried material being pelletized 29. In the case of animal feed, the molasses may enhance both the palatability and carbohydrate content of the animal feed. In the case of biomass fuel the molasses 21 may enhance the energy content and combustibility of the material. Excess molasses 30 may be sold as a food or energy source.

### EXAMPLES

Citrus waste was subjected to the basic process described generally above. The fermentation residue, distillers peels or whole stillage, was then dried and analyzed.

Results are shown for the analysis of feed with 10% moisture for: (1) Standard CPP, the feed was made exclusively from unprocessed biomass; (2) Distillers Peels 100%, where the feed is made exclusively from biomass processed to make ethanol; and (3) Distillers Peel 60%, where the feed is made from 60% of biomass processed to make ethanol and 40% unprocessed biomass.

| | Standard | Distillers | Distillers |
|---|---|---|---|
| | CPP | Peels 100% | Peels 60% |
| Carbohydrate (CHO) | 63% | 57% | 59% |
| Fiber | 11% | 10% | 10% |
| Protein | 6% | 8% | 8% |
| Fat | 3% | 5% | 4% |
| Ash | 7% | 10% | 9% |
| Water | 10% | 10% | 10% |
| Total | 100% | 100% | 100% |
| CHO + Fiber + | 83% | 80% | 81% |
| Fat + Protein | | | |

There is a minimum degradation in nutritional value as a larger percentage of processed biomass was used in making the feed. The compositional analysis shows that distillers peel made through the invention is of similar nutritional value to Standard CPP. In the feed made exclusively from biomass processed to make ethanol, there is an approximately 40% decrease in the mass of feed at 10% moisture which is produced, when compared to the amount produced using only unprocessed biomass.

By varying percentage of processed and unprocessed biomass used by the invention to make the animal feed or bio fuel, as well as the heat, pressure and timing of the reactions, a mixed-biomass pellet can be made that generates lower VOCs emissions during the manufacturing process, but is comparable in nutritional content, physical characteristics and shelf life to Standard CPP.

What is claimed is:

1. A biomass comprising:
Carbohydrates between about 55% to about 65%;
Fiber between about 8% to about 13%;
Protein between about 4% to about 10%;
Fat between about 1% to about 7%;
Ash between about 5% to about 12%; and
Water between about 8% to about 12%;
wherein the biomass is produced from an ethanol whole stillage with the use of a press aid.

2. The biomass according to claim 1, wherein the biomass serves as an animal feed.

3. The biomass according to claim 1, wherein the biomass serves as a biofuel.

4. The biomass according to claim 1, wherein the press aid is selected from the group consisting of wood, straw, bagasse, pulp and cellulose.

5. A biomass comprising:
Carbohydrates between about 55% to about 65%;
Fiber between about 8% to about 13%;
Protein between about 4% to about 10%;
Fat between about 1% to about 7%;
Ash between about 5% to about 12%; and
Water between about 8% to about 12%.
wherein the biomass is produced by the addition of unfermented citrus waste to an ethanol whole stillage.

6. The biomass according to claim 5, wherein the allocation of unfermented citrus waste in the production of the biomass is between about 90% to about 40%.

7. The biomass according to claim 5, wherein the biomass serves as an animal feed.

8. The biomass according to claim 5, wherein the biomass serves as a biofuel.

9. A method for producing a biomass, the method comprising (a) providing an ethanol whole stillage; (b) adding a press aid to the ethanol whole stillage to form a wet biomass; and (c) pressing the wet biomass to provide a pressed biomass.

10. The method according to claim 9, further comprising centrifuging the ethanol whole stillage before the press aid is added.

11. The method according to claim 9, wherein pressing comprises the use of a mechanical press.

12. The method according to claim 11, further comprising drying the pressed biomass to produce a dry biomass.

13. The method according to claim 12 wherein the ethanol whole stillage is derived from citrus peel waste that has undergone saccharification and fermentation.

14. A method for producing a biomass, the method comprising (a) providing an ethanol whole stillage; (b) adding unfermented citrus waste to the ethanol whole stillage to form a mixture of fermented and unfermented biomass; and (c) pressing the mixture to provide a mixed biomass.

15. The method according to claim 14, further comprising centrifuging the ethanol whole stillage before the unfermented citrus waste is added.

16. The method according to claim 14, wherein pressing comprises the use of a mechanical press.

17. The method according to claim 14, further comprising drying the mixed biomass produce a dry mixed biomass.

18. The method according to claim 17, wherein the drying results in reduced emissions of VOCs compared to emissions resulting from the preparation of CPP.

19. The method according to claim 17 wherein the ethanol whole stillage is derived from citrus peel waste that has undergone saccharification and fermentation.

20. The products and processes described herein.
